(19) United States
(12) Patent Application Publication
(10) Pub. No.: US 2023/0393124 A1
(43) Pub. Date: Dec. 7, 2023

(54) Methods and Systems for Quantitative Detection of SARS-CoV-2 Antibodies Using Dried Samples

(71) Applicant: Laboratory Corporation of America Holdings, Burlington, NC (US)

(72) Inventors: Russell Philip Grant, Chapel Hill, NC (US); Bradley B. Collier, Chapel Hill, NC (US)

(21) Appl. No.: 18/205,255

(22) Filed: Jun. 2, 2023

Related U.S. Application Data
(60) Provisional application No. 63/348,755, filed on Jun. 3, 2022.

(51) Int. Cl. G01N 33/533; G16H 10/40

(52) U.S. Cl. CPC G01N 33/533; G16H 10/40

(57) ABSTRACT

In certain aspects, disclosed are methods and systems for detecting SARS-CoV-2 analytes in dried samples, as for example, dried blood spots. For example, disclosed are methods for measuring an antibody to SARS-CoV-2 in a dried sample that include the steps of: (a) obtaining a dried sample from a subject; (b) extracting the SARS-COV-2 antibody from the dried sample; and (c) detecting the SARS-COV-2 antibody extracted from the dried sample. In certain embodiments, the method is semi-quantitative. The method may, in certain embodiments, further comprise obtaining measurements from an individual over a period of time to follow the titer of SARS-CoV-2 antibody in the individual. For example, the titer may be followed in the individual for at least 19 weeks or longer.

### RELATED APPLICATIONS

This application claims priority to U.S. Provisional Patent Application No. 63/348,755 filed Jun. 3, 2022. The disclosure of U.S. Provisional Patent Application 63/348,755 is incorporated by reference herein in its entirety.

### FIELD

Disclosed are methods and systems for detecting analytes in dried samples, as for example, antibodies to SARS-CoV-2, in dried blood spots that can be self-collected by a subject.

### INTRODUCTION

SARS-CoV-2 is an enveloped, single-stranded RNA virus of the family Coronaviridae, genus Beta coronavirus. All coronaviruses share similarities in the organization and expression of their genome, which encodes 16 nonstructural proteins and the 4 structural proteins: spike protein (S), envelope protein (E), membrane protein (M), and nucleocapsid protein (N). Viruses of this family are of zoonotic origin. They cause disease with symptoms ranging from those of a mild common cold to more severe symptoms such as the Severe Acute Respiratory Syndrome (SARS), Middle East Respiratory Syndrome (MERS) and Coronavirus Disease 2019 (COVID-19). Other coronaviruses known to infect people include 229E, NL63, OC43 and HKU1. The latter are ubiquitous and infection typically causes common cold or flu-like symptoms (Su S, Wong G, Shi W, et al., Epidemiology, Genetic Recombination, and Pathogenesis of Coronaviruses, Trends Microbiol., 24(6):490-502 (2016); Zhu N, Zhang D, Wang W, et al., A Novel Coronavirus from Patients with Pneumonia in China, 2019, N. Engl. J. Med., 382(8):727-733 (2020).

Despite the implementation of several measures to slow the spread of the disease, severe acute respiratory syndrome coronavirus 2 (SARS-CoV-2) continues to be an international public health emergency due to rapid human-to-human transmission and prevalence of asymptomatic carriers (Qi, L. et al., Factors associated with the duration of viral shedding in adults with COVID-19 outside of Wuhan, China: A retrospective cohort study, Int. J. Infect. Dis. 96, 531-537 (2020); World Health Organization (WHO), Infection prevention and control of epidemic- and pandemic-prone acute respiratory infections in health care, WHO Guide 1, 1-156, (2014) available on the internet at who.int/publications/i/iteminfection-prevention-and-control-of-epidemic-and-pandemic-prone-acute-respiratory-infections-in-health-care). Initially, many countries implemented physical distancing protocols and/or lockdown restrictions (Ghaffari, A., Meurant, R. and Ardakani, A., COVID-19 serological tests. How well do they actually perform? Diagnostics 10, 453 (2020)). In addition, diagnostic tests were quickly developed and granted FDA emergency use authorization (EUA) in order to identify individuals with active SARS-CoV-2 infections (Wang, Y. C. et al., Current diagnostic tools for coronaviruses—From laboratory diagnosis to POC diagnosis for COVID-19, Bioeng. Transl. Med., 5, 1-10 (2020)). Although social distancing and diagnostic testing continue to be vital to ending the pandemic, the advent of SARS-CoV-2 vaccines provides a more robust means of limiting the spread of the virus (Wang et al., 2020). By triggering the body's natural immune response, vaccines initiate the creation of antibodies that can neutralize the virus upon infection and ultimately reduce the severity of infections as well as transmission of the virus (Alessandro, S. and Crotty, S., Adaptive immunity to SARS-CoV-2 and COVID-19, Ann. Oncol., 19-21 (2020)). Unfortunately, the lifespan of circulating SARS-CoV-2 antibodies and the requisite titer to yield protective immunity against SARS-CoV-2 is still unclear (Alessandro and Crotty, 2020; Khoury, D. S. et al., Neutralizing antibody levels are highly predictive of immune protection from symptomatic SARS-CoV-2 infection, Nat. Med. 27, 1205-1211 (2021)). These concerns are further confounded by potential immunological differences between immunization versus native infection and the evolutionary nature of the virus (i.e., virus variants) (Alessandro and Crotty, 2020).

As the outbreak has progressed, it has become clear that the full scope in number of people exposed to SARS-CoV-2 cannot be determined by molecular testing which is appropriate for active infections. Therefore, a widely available high throughput method for determining who has or who has not been previously infected is needed. Large population analysis is needed to determine the percent of the population who have been infected to assist in policy decisions for prevention of viral spreading. One potential method for large scale sample collection while still providing safe and effective testing for patients is by lancing a finger and applying blood to a blood spot card followed by shipping to a central laboratory for testing. Dried blood spot card (DBS) collection can be performed by a trained phlebotomist or self-collected in one's home. However, further investigation including studies based on regulatory guidance is required prior to utilization of DBS samples for at-home self-collection (U.S. Food & Drug Administration. Home Specimen Collection Serology Template for Fingerstick Dried Blood Spot, (2020) available on the internet at www/fda.gov/medical-devices/coronavirus-disease-2019-covid-19-emergency-use-authorizations-medical-devices/vitro-diagnostics-euas).

Thus, there is a need for methods and systems for monitoring SARS-CoV-2 infection via serological testing that can be accessed by a large population, as for example, using samples such as dried blood spots that can be self-collected at home.

### SUMMARY

Disclosed are methods and systems for detecting an analyte of interest in a biological sample. In certain embodiments, the analyte of interest is an antibody to SARS-CoV-2. In certain embodiments, the sample is a dried biological sample.

For example, in certain embodiments the method may comprise a method for measuring an analyte of interest in a dried sample comprising: (a) obtaining a dried sample from a subject; (b) extracting the analyte of interest from the dried sample; and (c) detecting the analyte of interest extracted from the dried sample. In certain embodiments, the analyte of interest is an analyte specific to SARS-CoV-2. For example, the analyte of interest may be an antibody to SARS-CoV-2. In certain embodiments, the sample is dried blood, or dried serum or dried plasma from blood.

Also disclosed are systems for performing the disclosed methods or any of the steps of the disclosed methods, and a computer-program product tangibly embodied in a non-transitory machine-readable storage medium, including instructions configured to perform any of the steps of the disclosed methods or run any part of the disclosed systems.

In certain embodiments, the methods and systems provide a more rigorous testing than previous tests. Additionally, and/or alternatively, embodiments of the methods and systems provide a simplified sample self-collection process, a simplified extraction process, and a reduction in the assay's reporting limit for DBS samples.

### BRIEF DESCRIPTION OF THE FIGURES

The present disclosure may be better understood by referencing the following non-limiting figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 illustrates a method in accordance with an embodiment of the disclosure.

FIG. 2 illustrates a general process by which a patient may self-collect a dried blood spot (DBS) sample at home, send it into the laboratory, and how an analyte in the sample is extracted and measured in the lab in accordance with an embodiment of the disclosure.

FIG. 3 illustrates a system in accordance with an embodiment of the disclosure.

FIG. 4 illustrates a computing system in accordance with an embodiment of the disclosure.

FIGS. 5A-5D illustrate a method in accordance with an additional embodiment of the disclosure. FIG. 5A shows serum antibody concentrations for self-collected dried blood spot (DBS) sample antibody levels. FIG. 5B shows serum antibody concentrations for professionally collected DBS sample antibody levels. FIG. 5C shows calculated serum concentrations for DBS concentrations (by dividing DBS results by 0.070) for self-collected samples. FIG. 5D shows calculated serum concentrations for DBS concentrations (by dividing DBS results by 0.070) for professionally collected samples.

FIGS. 6A-6B illustrate SARS-CoV-2 antibody levels measured from DBS samples according to various embodiments of the disclosure following pre-dose (Day 0) through receipt of second vaccine dose (Day 21) to day 28 (FIG. 6A) and through Day 132 (FIG. 6B). Y-axes are displayed logarithmically with the left axis representing calculated serum levels (by dividing DBS results by 0.070).

### DETAILED DESCRIPTION

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section or as used elsewhere herein prevails over the definition that is incorporated herein by reference.

Many modifications and other embodiments of the disclosed subject matter set forth herein will come to mind to one skilled in the art to which the disclosed subject matter pertains having the benefit of the teachings presented in the description. Therefore, it is to be understood that the disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Definitions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

The term “about” is used to indicate that a value includes the inherent variation of error for the device, the method being employed to determine the value, or the variation that exists among sample.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

As used herein, an analyte is a molecule or biological compound being analyzed either qualitatively (e.g., for identification) or quantitatively (e.g., to determine a relative or absolute amount).

As used herein, a “subject” or “individual” are used interchangeably and may comprise an animal. Thus, in some embodiments, a sample obtained from a subject is obtained from a mammalian animal, including, but not limited to a human or fetus, a dog, a cat, a horse, a rat, a monkey, and the like. In some embodiments, the biological sample is obtained from a human subject. In some embodiments, the subject is a patient, that is, a living person presenting themselves in a clinical setting for diagnosis, prognosis, or treatment of a disease or condition.

“Sample” or “patient sample” or “biological sample” or “specimen” are used interchangeably herein. Non-limiting examples of samples that may be dried for analysis with the disclosed systems and methods include, blood or a blood product (e.g., serum, plasma, or the like), urine, nasal swabs, a liquid biopsy sample (e.g., for the detection of cancer), or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue sampler or other liquid aspirate. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis.

The terms “labeled” and “labeled with a detectable agent or moiety” are used herein interchangeably to specify that an entity (e.g., a nucleic acid probe, antibody) can be measured by detection of the label (e.g., visualized, detection of radioactivity, fluorescence and the like) for example following binding to another entity (e.g., a nucleic acid, polypeptide). The detectable agent or moiety may be selected such that it generates a signal which can be measured and whose intensity is related to (e.g., proportional to) the amount of bound entity. A wide variety of systems for labeling molecules (e.g., antigens, antibodies, nucleic acids) are known in the art. A label or labeling moiety may be directly detectable (i.e., it does not require any further reaction or manipulation to be detectable, e.g., a chemiluminescent label or a fluorophore is directly detectable) or it may be indirectly detectable. i.e., it is made detectable through reaction or binding with another entity that is detectable such as but not limited to a hapten is detectable by immunostaining after reaction with an appropriate antibody comprising a reporter such as a fluorophore. Suitable detectable agents include, but are not limited to radionucleotides, fluorophores, chemiluminescent agents, microparticles, enzymes, colorimetric labels, magnetic labels, haptens, molecular beacons, aptamer beacons, and the like.

Any of a wide variety of detectable agents can be used in the practice of the disclosure. Suitable detectable agents or moieties include, but are not limited to: various ligands, radionucleotides; fluorescent dyes; a metal (e.g., ruthenium) having electrochemical properties or other chemiluminescent agents (such as, for example, acridinium esters, stabilized dioxetanes, and the like); bioluminescent agents; spectrally resolvable inorganic fluorescent semiconductor nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; digoxigenin; haptens; and proteins for which antisera or monoclonal antibodies are available. For example, in some embodiments a first antigen: antibody: second antigen complex is bound to a biotin-labeled electrode such that application of a voltage results in a chemiluminescent emission. In such embodiments, the detectable label may be ruthenium.

As used herein, “a semi-quantitative assay” or “quantitative assay refers to an assay that can determine the amount of an analyte, as for example within a particular range of upper and lower limits. This can be contrasted with “a qualitative assay” which is designed to detect the presence or absence of an analyte.

As used herein, “negative percent agreement” or “NPA” is the proportion of comparative/reference method negative results in which the test method result is negative.

As used herein, “positive percent agreement” or “PPA” is the proportion of comparative/reference method positive results in which the test method result is positive.

As used herein, “LOD” or “limit of detection” or is the lowest amount of analyte in a sample that can be detected with stated probability. Typically, LOD is expressed as the limit of blank (LOB) plus 1.645×SD (or 2×SD) of low sample measurements.

Also, as used herein, “LLOQ” or “Lower Limit of Quantitation” or “LOQ” or “Limit of Quantitation” is the lowest amount of analyte in a sample that can be quantitatively determined with a stated acceptable precision and accuracy.

As used herein, “limit of quantitation” or “LOQ” is the lowest amount of analyte in a sample that can be quantitatively determined with stated acceptable precision and accuracy.

As used herein, the term “ULOQ” or “upper limit of quantitation” or “upper range of quantitation” is the highest amount of analyte in a sample that can be quantitatively determined without dilution.

### Methods for Detection of SARS-CoV-2 Antibodies in Dried Samples

Disclosed are methods and systems for detecting an analyte of interest in a biological sample. In certain embodiments, the analyte of interest is an antibody to SARS-CoV-2. In certain embodiments, the sample is a dried biological sample.

For example, in certain embodiments the method may comprise a method for measuring an analyte of interest in a dried sample comprising: (a) obtaining a dried sample from a subject; (b) extracting the analyte of interest from the dried sample; and (c) detecting the analyte of interest extracted from the dried sample. In certain embodiments, the analyte of interest is an analyte specific to SARS-CoV-2. For example, the analyte of interest may be an antibody to SARS-CoV-2. In certain embodiments, the dried sample is dried blood, or dried serum or dried plasma from blood.

Additionally, and/or alternatively, in certain embodiments the method may comprise measuring an antibody to SARS-CoV-2 in a sample comprising: (a) obtaining a sample from a subject; (b) extracting the SARS-COV-2 antibody from the sample; and (c) detecting the SARS-COV-2 antibody extracted from the sample. In certain embodiments, the sample is blood or serum or plasma from blood. In certain embodiments, the sample is dried blood, or dried serum or dried plasma from blood.

In certain embodiments, the method may comprise using a Roche Elecsys Anti-SARS-CoV-2 S electrochemiluminescence immunoassay. The Roche Elecsys Anti-SARS-CoV-2 S electrochemiluminescence immunoassay has received emergency use authorization (EUA) for measurement of antibodies in venous serum and plasma samples (Roche Diagnostics, Elecsys Anti-SARS-CoV-2 S Elecsys Anti-SARS-CoV-2 S. (2020)). The Elecsys® Anti-SARS-CoV-2 S for use on the cobas e analyzer is an electrochemiluminescence immunoassay intended for qualitative and semi-quantitative detection of antibodies to SARS-CoV-2 spike (S) protein receptor binding domain (RBD) in human serum and plasma (e.g., lithium heparin, dipotassium-EDTA, tripotassium-EDTA, and sodium citrate). Or other immunoassays, as for example, the Roche Elecsys anti-SARS-CoV-2 immunoassay, the DiaSorin Liaison SARS-CoV-2 S1/S2 IgG assay, the DiaSorin Liaison SARS-CoV-2 IgM) assay, the Euroimmun anti-SARS-CoV-2 IgG ELISA, the Euroimmun anti-SARS-CoV-2 NCP IgG ELISA, the Epitope Diagnostics Novel Coronavirus COVID-19 IgG ELISA kit, or the Luminex xMAP SARS-CoV-2 Multi-Antigen assay may be used.

Thus, in certain embodiments, the analyte of interest is an antibody, and the antibody is measured using a sandwich assay employing a first antigen labeled with a detectable moiety and a second antigen labeled with a binding agent. In certain embodiments, the detectable moiety on the first antigen is an electrochemical moiety. The detectable moiety may, in certain embodiments, comprise an electrochemical moiety such as ruthenium. Or other detectable moieties, such as radiolabels, fluorescent labels, heavy isotopes, and the like, may be used. Additionally, and/or alternatively, the binding agent may comprise streptavidin. Or, other binding agents, such as secondary antibodies, receptor ligands, and the like, may be used.

The first and second antigens may be antigens that recognize the antibody. In certain embodiments, the first and second antigens may be the same antigen, but differentially labeled with a detectable moiety and a binding agent, respectively. In an embodiment, the antigens may be synthesized in vitro. For example, for the Roche Elecsys Anti-SARS-CoV-2 S electrochemiluminescence immunoassay, the first and second antigens are recombinant antigens that recognize SARS-COV-2 antibodies to the SARS-CoV-2 spike (S) protein. In certain embodiments, a ruthenium labeled first antigen: the subject SARS-CoV-2 antibody: streptavidin labeled second antigen complex is bound to a biotin labeled electrode such that application of a voltage results in a chemiluminescent emission from the ruthenium.

The method may be quantitative or semi-quantitative in nature. In certain embodiments, the limit of quantitation (LOQ) may be about 0.180 U/mL per dried blood spot (DBS) sample. Additionally, and/or alternatively, the upper range of quantitation may be about 250 U/mL DBS sample. In an embodiment, this range represents a calculated serum measurement range of about 2.6 to 3570 U/mL. In certain embodiments, the assigned U/mL are equivalent to Binding Antibody Units (BAU)/mL as defined by the first World Health Organization (WHO) International Standard for anti-SARS-Cov-2 immunoglobulin. In certain embodiments, the limit of blank (LOB) is about 0.111 U/mL per DBS sample. In certain embodiments, exogenous interferents have mean biases less than ±5.0% while most endogenous interferents (with the exception of excess protein) have a mean bias result less than ±10.0%.

The methods and systems may have a variety of clinical uses. For example, the method may further comprise obtaining measurements from an individual over a period of time to follow the titer of SARS-CoV-2 antibody in the individual. In certain embodiments, the titer is followed in the individual for at least 5, or 10, or 15, or 19, or 20 or 25 weeks or longer. In certain embodiments, a clinical cutoff value below the EUA approved assay's LOQ for serum and plasma can be implemented for DBS extracts, enabling assessment of categorical agreement between venous and DBS samples near the serum cutoff (0.8 U/mL). In certain embodiments (e.g., using the Roche Elecsys Anti-SARS-CoV-2 S electrochemiluminescence immunoassay as described herein), the DBS clinical cutoff for positivity is then set to ≥0.185 U/mL, where all results less than 0.185 are reported as negative.

The accuracy and precision of the disclosed methods and systems may be verified by a variety of techniques. In certain embodiments, the results are compared to RT-PCR detection of SARS-CoV-2 nucleic acid. In certain embodiments, compared to RT-PCR results, qualitative categorical agreement is 99.1% with an NPA and PPA of 100.0% and 97.0%.

The disclosed methods and systems may be used to measure a variety of analytes. In an embodiment, the analyte is specific to SARS-CoV-2. For example, in some embodiments, the SARS-CoV-2 specific analyte is an antibody that recognizes the SARS-CoV-2 spike protein. Or the antibody may recognize other SARS-CoV-2 proteins. Or the analyte may be a protein or antigen specific to a pathogen of interest. For example, in some embodiments, the SARS-CoV-2 specific analyte is an antigen or other protein specific to SARS-CoV-2. Or, the analyte may be a nucleic acid. For example, in some embodiments, the SARS-CoV-2 specific analyte is a nucleic acid specific to SARS-CoV-2. Or, analytes from other viruses, bacteria, or other sources or analytes of interest may be analyzed.

The utilization of vaccines to fight the spread of SARS-CoV-2 has led to a growing need for expansive serological testing. To address this, an EUA approved immunoassay for detection of antibodies to SARS-CoV-2 in venous serum samples for use with dried blood spot (DBS) samples such as the disclosed methods is needed. As disclosed herein, results from self-collected DBS samples may demonstrate a 98.1% categorical agreement to venous serum with a correlation (R) of 0.9600 while professionally collected DBS samples may demonstrate a categorical agreement of 100.0% with a correlation of 0.9888 to venous serum. Additionally, in certain embodiments, studies performed to stress different aspects of at-home DBS collection, including shipping stability, effects of interferences, and other sample-specific robustness studies may demonstrate a categorical agreement of at least 95.0% and a mean bias less than ±20.0%. In certain embodiments, the disclosed methods have the ability to track antibody levels following vaccination with the BioNTech/Pfizer vaccine using serial self-collected DBS samples from pre-dose (Day 0) out to 19 weeks.

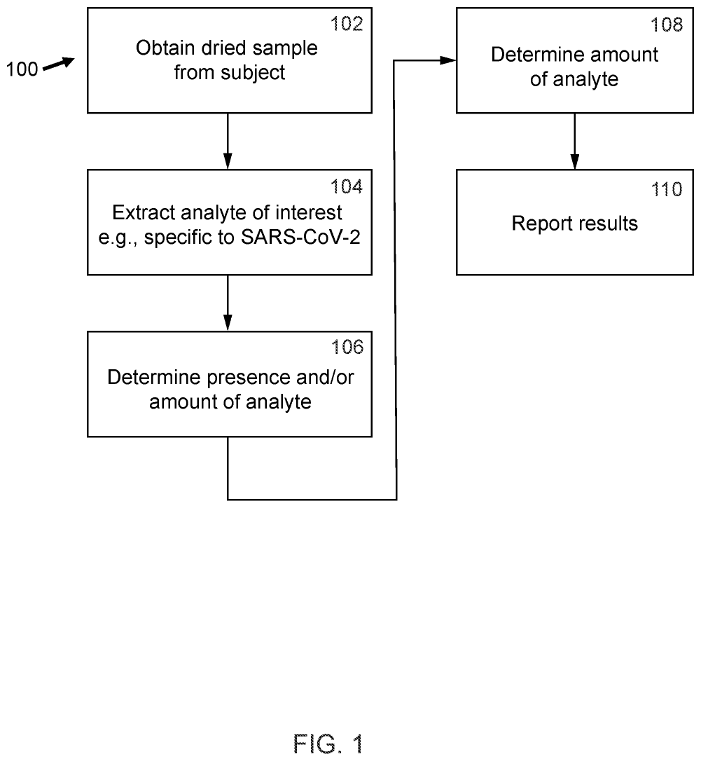

FIG. 1 illustrates an embodiment of the disclosed methods. Thus, as illustrated in FIG. 1, the method 100 may comprise the step of obtaining a dried sample from a subject 102. In certain embodiments, the sample is a dried blood spot or a dried plasma sample. In an embodiment, the dried blood spot (DBS) may be obtained by a subject taking a small sample of his or her own blood. Sampling may be by a medical professional, the subject requesting testing, or another individual with the subject's permission. Thus, in an embodiment, the DBS may be procured by a subject in his or her own home, without the need to visit a health care professional or commercial testing site. Or, when the subject is not able to take their own sample (e.g., a child or a non-human subject) another individual may procure the DBS. In an embodiment, proper dosing of the DBS card is critical to the extraction and measurement of the sample. For example, blood (i.e. from a lanced finger) may be added dropwise to a DBS card. In an embodiment, blood is applied to a plurality of application areas on a card or other solid support (e.g., 5 circles) that may be delineated (e.g., by dashed lines or other markings) on the solid support. The blood may be applied until blood fills these predefined regions. In certain embodiments, enough sample is required to obtain two (2) punches of approximately ¼″ diameter completely saturated with blood. Or, other sample sizes (volumes) may be used.

Generally, any type of substrate suitable for depositing a liquid sample for drying and subsequent extraction of an analyte of interest may be used. For example, Perkin Elmer 226, Whatman 903, or Eastern Business Forms 903 dried blood spot cards can be used. Blood spots may be obtained on a card and dried using instructions provided with a blood collection kit. In certain embodiments, samples are dried for a minimum of 3 hours. In an embodiment, samples returned to laboratory can be tested up to 36 days from collection as long as sample remains in an appropriate container (e.g., a Blood Sample Return bag) or other appropriate packaging.

As shown in FIG. 1, the method may further comprise extracting the analyte of interest from the dried sample 104. For example, in certain embodiments, the method may comprise extracting the analyte specific to SARS-CoV-2 from a DBS. The method of extraction may be varied depending upon the technique used to measure the analyte of interest. In certain embodiments, and as discussed in detail herein, the extraction reagent and/or volume may be modified from that which is typically used as is needed to optimize recovery and measurement of the analyte of interest from a dried sample as compared to plasma, blood or other liquid samples.

As further illustrated in FIG. 1, the method may comprise determining the presence and/or amount of the analyte in the sample 106. The disclosed methods and systems may be used with a variety of analytical techniques. In certain embodiments, the analyte of interest is an antibody, such as an antibody that recognizes the SARS-CoV-2 spike protein or other SARS-CoV-2 proteins. In such embodiments, the antibody of interest may be measured using a sandwich assay. The sandwich assay may employ a first antigen labeled with a detectable moiety and a second antigen labeled with a binding agent.

In one embodiment, the analytical technique comprises a Roche Elecsys Anti-SARS-CoV-2 S electrochemiluminescence immunoassay assay as disclosed in detail herein or a similar assay. For example, for the Roche Elecsys Anti-SARS-CoV-2 S electrochemiluminescence immunoassay, the first and second antigens are recombinant antigens that recognize SARS-COV-2 antibodies to the SARS-CoV-2 spike (S) protein. For example, SARS-CoV-2 antibody extracted from a dried sample, such as a DBS or dried plasma, may then be measured by detection of SARS-CoV-2 antibody bound to the first and second antigen. The detectable moiety may, in certain embodiments, comprise an electrochemical moiety such as ruthenium. Or other detectable moieties, such as radiolabels, fluorescent labels, heavy isotopes, and the like, may be used. Additionally, and/or alternatively, the binding agent may comprise streptavidin. Or, other binding agents, such as secondary antibodies, receptor ligands, and the like, may be used. In certain embodiments, a ruthenium labeled first antigen: subject SARS-CoV-2 antibody: streptavidin labeled second antigen complex is bound to a biotin labeled electrode such that application of a voltage results in a chemiluminescent emission from the ruthenium.

As further illustrated in FIG. 1, in certain embodiments, the amount of the SARS-CoV-2 antibody is determined 108. In certain embodiments, the antibody level is calculated to incorporate dilution of the sample that occurs during extraction of the SARS-CoV-2 specific analyte from the dried blood spot. In such embodiments, and as described in detail herein, the measured value accounts for dilution to provide a normalized value. In an embodiment, a normalized value is a dilution corrected value equivalent to venous liquid serum or plasma results. For example, in certain embodiments, the limit of quantitation (LOQ) for SARS-CoV-2 antibodies is about 0.180 U/mL per DBS sample. Additionally, and/or alternatively, the upper range of quantitation for SARS-CoV-2 antibodies may be about 250 U/mL DBS sample. In certain embodiments, the limit of blank (LOB) may be about 0.111 U/mL per DBS sample.

Referring again to FIG. 1, at this point, the results may be reported to the subject or their healthcare provider 110. Such results may be used to determine if retesting and/or continued monitoring of the subject is advised.

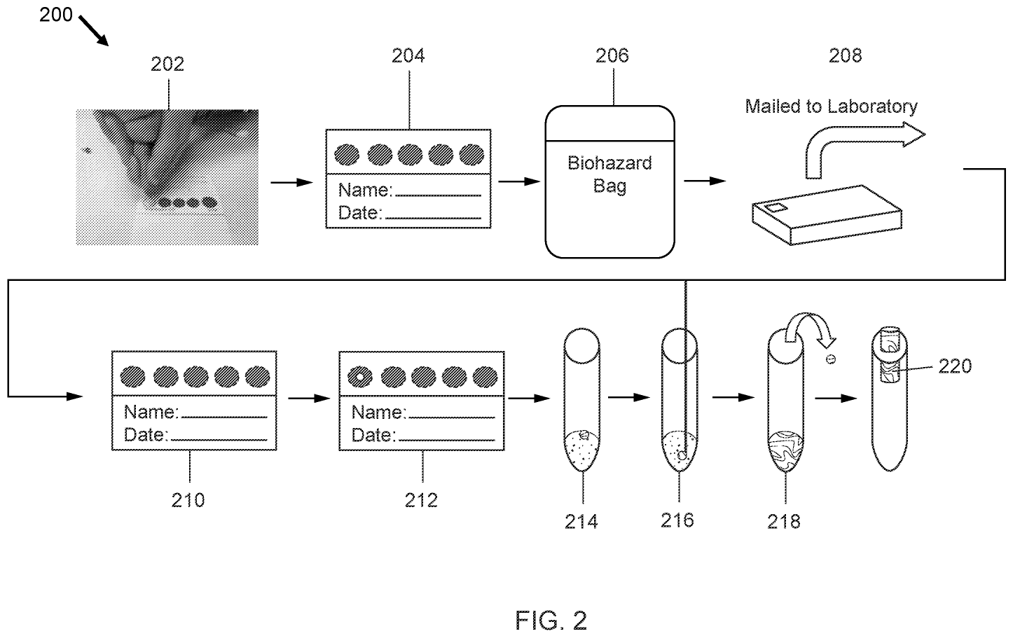

FIG. 2 shows another illustration of an embodiment of certain of the steps of a disclosed method 200. Thus, as shown in FIG. 2 an individual may apply drops of blood to a card 202 that includes the subject's name and the date the sample was procured 204. The card may then be inserted into a Blood Sample Return bag or other appropriate packaging (e.g., biohazard bag) 206 and then mailed to a testing laboratory 208. Upon arrival at the testing laboratory, the card may be recorded 210 and then a portion of a single DBS sample removed for processing 212. The DBS sample may be placed in a tube containing a diluent (e.g., buffer) 214 and submerged in the diluent using an applicator 216 and incubated (e.g., at room temperature) to elute the blood cells from the card 218. At this point, a portion of the blood sample may be transferred to a new container (e.g., a microcup) for measurement of the analyte of interest 220.

### Systems for Detection of SARS-CoV-2 Analytes in Dried Samples

Also disclosed are systems for performing any of the steps of the disclosed methods and computer-implemented instructions for performing any of the steps of the disclosed methods or running any of the parts of the disclosed systems.

For example, disclosed is a system for measuring an analyte in a dried sample comprising: (a) a component or station for obtaining a dried sample from a subject; (b) a component or station for extracting the analyte from the sample; and (c) a component or station detecting the analyte extracted from the sample. In an embodiment, the analyte may be an antibody. For example, the antibody may an antibody to SARS-CoV-2. In some embodiments, the antibody may recognize the SARS-CoV-2 spike protein.

In certain embodiments, the antibody is a SARS-COV-2 antibody and the step of detecting the SARS-COV-2 antibody is performed using a Roche Elecsys Anti-SARS-CoV-2 S electrochemiluminescence immunoassay. For the Roche Elecsys Anti-SARS-CoV-2 S electrochemiluminescence immunoassay, the first and second antigens are recombinant antigens that recognize SARS-COV-2 antibodies to the SARS-CoV-2 spike (S) protein. In certain embodiments, the first and second antigens may be the same antigen, but differentially labeled with a detectable moiety and a binding agent, respectively. For example, SARS-CoV-2 antibody extracted from a dried sample, such as a DBS or dried plasma, may be measured by detection of SARS-CoV-2 antibody bound to the first and second antigen. The detectable moiety may, in certain embodiments, comprise an electrochemical moiety such as ruthenium. Or other detectable moieties, such as radiolabels, fluorescent labels, heavy isotopes, and the like, may be used. Additionally, and/or alternatively, the binding agent may comprise streptavidin. Or, other binding agents, such as secondary antibodies, receptor ligands, and the like, may be used. In certain embodiments, a ruthenium labeled first antigen: subject SARS-CoV-2 antibody: streptavidin labeled second antigen complex is bound to a biotin labeled electrode such that application of a voltage results in a chemiluminescent emission from the ruthenium.

As disclosed herein, a variety of samples may be used with the disclosed systems. In certain embodiments, the sample may be dried blood, or dried serum or dried plasma. In certain embodiments, the dried sample is a dried blood spot (DBS).

The disclosed systems may provide results that are sensitive and quantitative. Thus, in certain embodiments, the method is semi-quantitative or quantitative. In certain embodiments, the LOD and/or LOQ is 0.180 U/mL per DBS sample and/or the upper range of detecting or upper limit of quantitation (ULOQ) is 250 U/mL DBS sample. In an embodiment, this DBS range represents a calculated serum measurement range of about 2.6 to 3570 U/mL. In certain embodiments, the limit of blank (LOB) is about 0.111 U/mL per DBS sample. Also in certain embodiments, the DBS clinical cutoff for positivity is set to ≥0.185 U/mL, such that results less than 0.185 are reported as negative.

In certain embodiments, the system may be used to monitor an individual over a period of time, e.g., to follow the titer of SARS-CoV-2 antibody in the individual. In certain embodiments, the titer may be followed in the individual for at least 5, or 10, or 15, or 19, or 20 or 25 weeks or more.

In certain embodiments, the results are compared to RT-PCR detection of SARS-CoV-2 nucleic acid. For example, in certain embodiments, compared to RT-PCR results for the detection of SARS-CoV-2 nucleic acid, qualitative categorical agreement for detection of antibodies to SARS-CoV-2 is about 99.1% with an negative percent agreement (NPA) of 100.0% and/or positive percent agreement (PPA) of at least 97.0%. Also, in certain embodiments of the disclosed systems, exogenous interferents have mean biases less than ±5.0% while most endogenous interferents have a mean bias result less than ±10.0%.

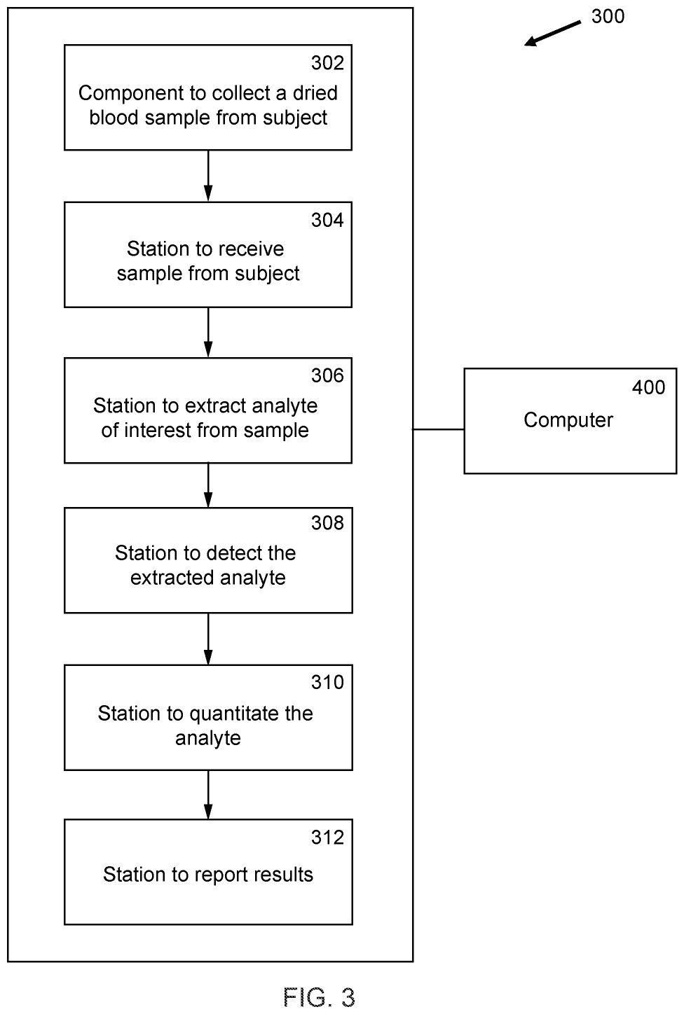

Thus, as illustrated in FIG. 3 the system 300 may comprise a station or component to collect a dried blood sample from a subject 302. In certain embodiments, the sample is a dried blood spot or a dried plasma sample. In an embodiment, the dried blood spot (DBS) may be obtained by a subject using a solid support (e.g., DBS card) by taking a small sample of his or her own blood. In an embodiment, proper dosing of the DBS card is critical to the extraction and measurement of the sample. For example, using a DBS card of the system, blood (i.e., from a lanced finger) may be added dropwise to the DBS card. The blood may be applied to a plurality of application areas on a card or other solid support (e.g., 5 circles) that may be delineated (e.g., by dashed lines or other markings) on the solid support. The blood may be applied until blood fills these predefined regions. In certain embodiments, two (2) punches of approximately ¼″ diameter completely saturated with blood is sufficient for the analysis. Or, other sample sizes (volumes) may be used.

The system may further comprise a station or component 304 for receiving a sample (e.g., a DBS card). For example, upon arrival at the testing laboratory, a subject's DBS card may be recorded and indicia required for reporting the results entered into a database. The card may then be stored (e.g., at room temperature or in a refrigerator or freezer) for further processing.

The system may further comprise a station or component for extracting the analyte of interest from the sample 306. Thus, in certain embodiments, a portion of a DBS sample may be placed in a tube containing a diluent (e.g., buffer), submerged in the diluent using an applicator, and incubated (e.g., at room temperature) to elute the blood cells from the card. At this point, a portion of the blood sample may be transferred to a new container (e.g., a microcup) for measurement of the analyte of interest.

Also, the system may comprise a station or component for detecting the analyte 308. A variety of analytical techniques may be used. In one embodiment, the system comprises a Roche Elecsys Anti-SARS-CoV-2 S electrochemiluminescence immunoassay. For example, the SARS-CoV-2 antibody may be measured using a first antigen labeled with a detectable moiety and a second antigen labeled with a binding agent. For example, SARS-CoV-2 antibody extracted from a dried sample, such as a DBS or dried plasma, may then be measured by detection of SARS-CoV-2 bound to the first and second antigen. The detectable moiety may, in certain embodiments, comprise an electrochemical moiety such as ruthenium. Or other detectable moieties, such as radiolabels, fluorescent labels, heavy isotopes, and the like, may be used. Additionally, and/or alternatively, the binding agent may comprise streptavidin. Or, other binding agents, such as secondary antibodies, receptor ligands, and the like, may be used. For example, in certain embodiments, a ruthenium labeled first antigen: SARS-CoV-2 antibody: streptavidin labeled second antigen complex is bound to a biotin labeled electrode (by binding of the streptavidin to biotin) such that application of a voltage results in a chemiluminescent emission.

The system may comprise a station or component for determining the amount of the analyte 310 based on the analytical assay 308. For example, an electrochemiluminescence assay may provide a quantitative measurement of antibody (or antigen) based on the change in electrochemiluminescence (ECL) signal before and after the immunoreaction. For the Elecsys Anti-SARS-CoV-2 S electrochemiluminescence immunoassay the amount of SARS-CoV-2 may be quantified by measuring the level of the ECL signal (from ruthenium) present on the first antigen (of the sandwich) upon binding of the complex to a biotin-labeled electrode via the streptavidin-labeled second antigen.

The system may further comprise a station and/or component to report the results 312.

In some embodiments, the system 300 further comprises a computer 400 and/or a data processor configured to run any of the stations of the system. As disclosed herein, in certain embodiments, the system may comprise one or more computers, and/or a computer product tangibly embodied in a non-transitory computer readable storage medium containing instructions which, when executed on the one or more data processors, cause the one or more data processors to perform actions for performing any of the steps of the methods or implementing the systems or portions of the systems (e.g., components and/or stations) of any of embodiments disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines. For example, the system may comprise a computer and/or computer-program product tangibly embodied in a non-transitory machine-readable storage medium for determining the measured analyte values. Thus, in certain embodiments, the system may comprise components to quantify the measurement of an analyte of interest. Also, the system may comprise components to perform statistical analysis of the data.

Thus, also disclosed herein is a computer (e.g., data processor) and/or a computer-program product tangibly embodied in a non-transitory machine-readable storage medium, including instructions configured to run any of the stations/components of the system and/or perform a step or steps of the methods of any of the disclosed embodiments. In one embodiment, the system comprises a computer and/or a computer-program product tangibly embodied in a non-transitory machine-readable storage medium, including instructions configured to identify the presence of and/or determine the amount of SARS-CoV-2 antibody in a dried sample from a subject. For example, in an embodiment, disclosed is a computer and/or a computer-program product tangibly embodied in a non-transitory machine-readable storage medium, including instructions configured to measure an antibody to SARS-CoV-2 in a dried sample comprising: (a) obtaining a dried sample from a subject; (b) extracting the SARS-COV-2 antibody from the dried sample; and (c) detecting the SARS-COV-2 antibody extracted from the dried sample. Or in an embodiment, disclosed is a computer or a computer-program product tangibly embodied in a non-transitory machine-readable storage medium, including instructions configured to control or run at least in part at least one of: (a) a component for obtaining a dried sample from a subject; (b) a component or station for extracting the antibody from the sample; and (c) a component or station detecting the antibody extracted from the sample.

In an embodiment the analyte may be an antibody. In certain embodiments, the antibody is an antibody to SARS-CoV-2. In some embodiments, the antibody may recognize the SARS-CoV-2 spike protein.

In certain embodiments, the antibody is a SARS-COV-2 antibody and the step of, or component for, detecting the SARS-COV-2 antibody is performed using a Roche Elecsys Anti-SARS-CoV-2 S electrochemiluminescence immunoassay. The Roche Elecsys Anti-SARS-CoV-2 S electrochemiluminescence immunoassay uses first and second antigens that are recombinant antigens that recognize SARS-COV-2 antibodies to the SARS-CoV-2 spike (S) protein. For example, SARS-CoV-2 antibody extracted from a dried sample, such as a DBS or dried plasma, may then be measured by detection of SARS-CoV-2 antibody bound to the first and second antigen. In certain embodiments, the first and second antigens may be the same antigen, but differentially labeled with a detectable moiety and a binding agent, respectively. The detectable moiety may, in certain embodiments, comprise an electrochemical moiety such as ruthenium. Or other detectable moieties, such as radiolabels, fluorescent labels, heavy isotopes, and the like, may be used. Additionally, and/or alternatively, the binding agent may comprise streptavidin. Or other binding agents, such as secondary antibodies, receptor ligands, and the like, may be used. In certain embodiments, a ruthenium labeled first antigen: subject SARS-CoV-2 antibody: streptavidin labeled second antigen complex is bound to a biotin labeled electrode such that application of a voltage results in a chemiluminescent emission from the ruthenium.

As disclosed herein, a variety of samples may be assessed using the computer and/or computer-program product tangibly embodied in a non-transitory machine-readable storage medium. In certain embodiments, the sample may be dried blood, or dried serum or dried plasma. In certain embodiments, the dried sample is a dried blood spot (DBS).

The disclosed computer and/or computer-program product tangibly embodied in a non-transitory machine-readable storage medium may provide results that are sensitive and quantitative. Thus, in certain embodiments, detection of the SARS-CoV-2 antibody is semi-quantitative or quantitative. In certain embodiments, the LOD and/or LOQ is 0.180 U/mL per DBS sample and/or the upper range of detecting or upper limit of quantitation (ULOQ) is 250 U/mL DBS sample. In an embodiment, this DBS range represents a calculated serum measurement range of about 2.6 to 3570 U/mL. In certain embodiments, the limit of blank (LOB) is 0.111 U/mL per DBS sample. Also in certain embodiments, the DBS clinical cutoff for positivity is set to ≥0.185 U/mL, such that results less than 0.185 are reported as negative.

In certain embodiments, the computer and/or computer-program product tangibly embodied in a non-transitory machine-readable storage medium may be used to monitor an individual over a period of time, e.g., to follow the titer of SARS-CoV-2 antibody in the individual. In certain embodiments, the titer may be followed in the individual for at least 5, or 10, or 15, or 19, or 20 or 25 weeks or more.

In certain embodiments, the results are compared to RT-PCR detection of SARS-CoV-2 nucleic acid. For example, in certain embodiments, compared to RT-PCR results for the detection of SARS-CoV-2 nucleic acid, qualitative categorical agreement for detection of antibodies to SARS-CoV-2 is about 99.1% with an negative percent agreement (NPA) of 100.0% and/or positive percent agreement (PPA) of at least 97.0%. Also, in certain embodiments of the disclosed computer and/or computer-program product tangibly embodied in a non-transitory machine-readable storage medium, exogenous interferents have mean biases less than ±5.0% while most endogenous interferents have a mean bias result less than ±10.0%.

Additionally, and/or alternatively, the computer and/or computer program product may comprise instructions and/or procedures for defining the measured analyte values. As noted above, this may depend on the sensitivity of the assay and/or the prevalence of the analyte of interest and/or pathogen from which it is derived in the population. Such results can then be reported to the subject providing the DBS sample or their health care provider.

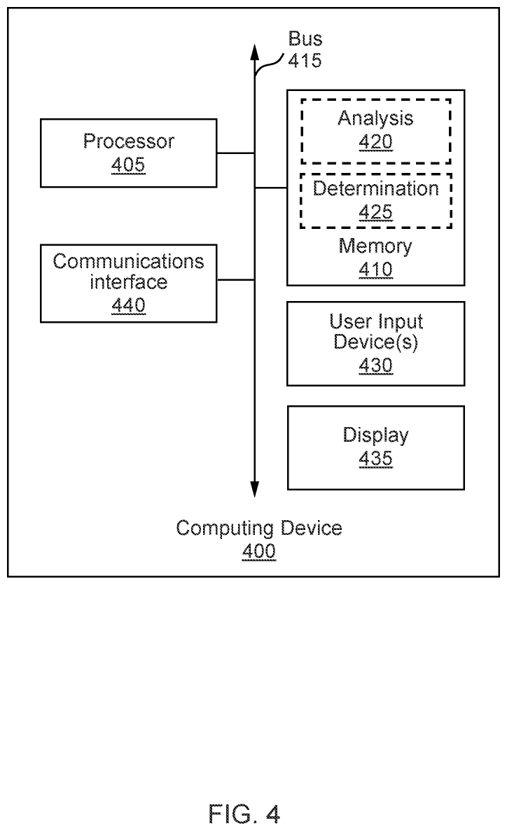

FIG. 4 shows a block diagram of an analysis system 400 used for detection and/or quantification of an analyte from a dried sample. As illustrated in FIG. 4, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 4, modules, engines, or components are shown for implementing the methods or running any of the systems of the disclosure.

Thus, FIG. 4 illustrates an example computing device 400 suitable for use with systems and the methods according to this disclosure. The example computing device 400 includes a processor 405 which is in communication with the memory 410 and other components of the computing device 400 using one or more communications buses 415. The processor 405 is configured to execute processor-executable instructions stored in the memory 410 to perform one or more methods or operate one or more stations for detecting antibodies to SARS-CoV-2 according to different examples, such as those in FIG. 1-3 or 5-6 or disclosed elsewhere herein. In this example, the memory 410 may store processor-executable instructions 425 that can analyze 420 results for sample as discussed herein.

The computing device 400 in this example may also include one or more user input devices 430, such as a keyboard, mouse, touchscreen, microphone, etc., to accept user input. The computing device 400 may also include a display 435 to provide visual output to a user such as a user interface. The computing device 400 may also include a communications interface 440. In some examples, the communications interface 440 may enable communications using one or more networks, including a local area network (“LAN”); wide area network (“WAN”), such as the Internet; metropolitan area network (“MAN”); point-to-point or peer-to-peer connection; etc.

Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### EXAMPLES

### Example 1—Assay Principle

The Roche Anti-SARS-CoV-2 S assay is to be used with the Roche e801 immunoassay module as part of a Roche Cobas 8000 instrument which can measure samples in Hitachi microcups. As the microcups have a listed dead volume of 50 μL compared to 100 μL for the standard sample cup, this reduces the volume requirement for measurement and influenced the volume of extraction buffer that was utilized (see below).

The assay for COVID-19 antibodies is based on the sandwich principle. During the first incubation, 30 μL of sample (increased from 12 μL for the EUA approved Roche Anti-SARS-CoV-2 S assay), a biotinylated SARS-CoV-2-specific recombinant antigen and SARS-CoV-2-specific recombinant antigen labeled with a ruthenium complex form a sandwich complex. Volumes of other reagents were also reduced in the DBS assay to keep the total volume with all reagents and sample consistent to the serum assay. After addition of streptavidin-coated microparticles, the complex becomes bound to the solid phase via interaction of biotin and streptavidin. The reaction mixture was aspirated into the measuring cell where the microparticles are magnetically captured onto the surface of the electrode. Unbound substances were then removed and application of a voltage to the electrode then induces chemiluminescent emission which is measured by a photomultiplier. The Roche Anti-SARS-CoV S assay uses the Roche Elecsys Anti-SARS-CoV-2 S assay kit (Roche 09289275190) containing both reagent and calibrator. Reagents and calibrators were used as received and were not modified for use.

### Example 2—Materials and Methods

A. Sample Extraction and Measurement

For extraction of DBS samples, two ¼″ diameter round hole punches were taken from regions of the DBS card that were saturated with blood (i.e. no white is visible on the punches) and placed into a single 16×75 mm polypropylene tube. Punches were then submerged in 150 μl of Roche Universal Diluent (07299001190) using a wooden applicator. Tubes were then placed on a micro plate shaker (VWR, 12620-926) at 240 rpm for one hour at room temperature (20-25° C.). Following extraction, remnant solution was squeezed out of the punches which were then discarded. The remaining extract (˜100 μL) was then transferred into a Hitachi microcup (system dead volume of 50 μL) for measurement on a Roche Cobas 8000 e801 immunoassay module.

Measurement of SARS-CoV-2 antibodies was performed using the Roche Elecsys Anti-SARS-CoV-2 S assay which has received EUA approval for the semi-quantitative measurement of total SARS-CoV-2 antibodies in serum and plasma samples. The lower numerical reporting limit was reduced from 0.400 U/mL (venous sample limit of quantitation) to 0.000 U/mL. This was necessary as samples obtained using DBS are diluted (˜tenfold) through the extraction process. By reducing the reporting limit, a lower LOQ for DBS extracts could be investigated and reduce the possibility of false negative results for patients with serum antibody results just above the serum clinical cutoff (0.800 U/mL) (Roche Diagnostics, Elecsys Anti-SARS-CoV-2 S Elecsys Anti-SARS-CoV-2 S (2020)). For example, a patient with serum antibody results of 3.00 U/mL would have a DBS result of 0.300 U/mL (assuming tenfold dilution and complete analyte recovery) which is less than the EUA approved assay LOQ (0.400 U/mL). No other EUA assay parameters were modified.

B. Clinical Agreement Studies

Paired venous serum and DBS samples were collected from individuals with previous COVID-19 infection (based on EUA approved RT-PCR diagnostic assays, n=36) as well as from presumed negative individuals (n=84). Donors previously infected with COVID-19 had nasopharyngeal samples collected and tested (using EUA approved methodologies for detection of SARS-CoV-2) between October and December 2020. All DBS and serum samples for this study were collected in January 2021.

Venous samples were obtained using traditional venipuncture techniques while DBS samples were obtained following sterilization of the fingertip with an alcohol pad and lancing the finger with a high flow contact-activated lancet (BD #366594). After wiping the first drop of blood with a gauze pad, blood was applied to the DBS card (Eastern Business Forms 903™ Dried Blood Spot Card, 10550021) to fill all five spots (˜50 L/spot). No instructions were provided regarding milking or squeezing of the finger after the fingerstick. Following collection, DBS samples were dried for three hours at room temperature (20-25° C.) and then placed into a plastic specimen pouch (without desiccant) for storage until testing. For self-collection of DBS samples, all donors provided samples in a home-like setting using detailed instructions for use to assist with the process described above. To rule out potential active asymptomatic infection for presumed negative donors, a self-collected nasal swab was procured concurrently and analyzed using Labcorp's EUA approved RT-PCR assay for SARS-COV-2 (U.S. Food & Drug Administration. In Vitro Diagnostics EUAs-Molecular Diagnostic Tests for SARS-CoV-2|FDA, available on the internet at www.fda.gov/medical-devices/coronavirus-disease-2019-covid-19-emergency-use-authorizations-medical-devices/in-vitro-diagnostics-euas-molecular-diagnostic-tests-sars-cov-2 (2021)).

C. Creation of Contrived Blood Samples

For specific validation studies, contrived blood samples were created due to the difficulty of obtaining DBS samples at specific concentrations and in large volumes. These samples were generated by mixing venous serum (screened for SARS-CoV-2 antibodies) with packed red blood cells to create samples with 40% hematocrit. Red blood cells were obtained intravenously from a seronegative donor with Type O blood using EDTA tubes. After mixing, the contrived blood samples were pipetted onto DBS cards (˜50 μL/spot) which were then allowed to dry for 3 h at room temperature prior to storage. For many studies (specifically those regarding sample and assay robustness), contrived blood samples were created such that approximately 25% of the samples utilized were negative samples within 5 times the DBS assay cutoff, 50% were positive samples within 5 times the cutoff, and 25% were greater than 5 times the cutoff. Contrived DBS samples were not utilized for the clinical correlation study.

### Example 3—Results and Discussion

A. Assessment of DBS Analytical Range and Imprecision

DBS Limit of Blank (LOB)

In order to assess the detection capability of the assay with DBS extracts, guidance from CLSI EP17-A2 was utilized (Clinical and Laboratory Standards Institute, EP17-A2 Evaluation of Detection Capability for Clinical Laboratory Measurement Procedures; Approved Guideline, (2012)). Two separate reagent lots were used to make 96 blank measurements on 6 contrived blood samples over a 4-day period. Two results were not included in the data analysis for each lot as the z-score for each of these results with respect to the remaining results were greater than 4.7 for both reagent lots. Using the mean and standard deviation of the remaining blank results (n=94) as well as a normal distribution multiplier, the LOB for DBS extracts was determined to be 0.111 U/mL.

DBS Limit of Detection (LOD)

The limit of detection (LOD) was assessed using 5 contrived blood samples in accordance with CLSI EP05-A3 and CLSI EP17-A2 guidance (Clinical and Laboratory Standards Institute, EP17-A2 (2012); Clinical and Laboratory Standards Institute, EP05-A3 Evaluation of precision of quantitative measurement procedures, Clinical and Laboratory Standards Institute vol. 34 (2014)). The samples utilized had mean results (across two reagent lots) that were expected to be close to the clinical cutoff (0.146-0.531 U/mL). Pooled standard deviations were calculated for the two different reagent lots using CLSI EP17-A2 guidance (Clinical and Laboratory Standards Institute, EP17-A2, (2012)). The first reagent lot produced a pooled standard deviation of 0.0419 U/mL while the second demonstrated a standard deviation of 0.0346 U/mL. Using the larger standard deviation (0.0419 U/mL), a normal distribution multiplier, and the reported LOB (0.111 U/mL), the LOD for DBS extracts was determined to be 0.180 U/mL.

DBS Limit of Quantitation (LOQ)

Measurements to determine the limit of quantitation (LOQ) of DBS extracts utilized 14 contrived blood samples covering an appropriate range of concentrations (0.0528-0.648 U/mL). These samples were extracted and measured in triplicate over a five-day period (15 total measurements for each level) using two different reagent lots on a single instrument. For this study, the target CV and bias were set to 25.0% based on FDA guidance for ligand binding assays at the lower limit of quantitation (Food and Drug Administration, Bioanalytical method validation guidance for industry, Food Drug Adm., (2018)). Following collection of data, the imprecision profiles were analyzed using the Limit of Quantitation module in EP Evaluator. These results indicated an LOQ of 0.0873 U/mL for the first reagent lot while the second reagent lot demonstrated an LOQ of 0.0736 U/mL. In addition, acceptable biases less than ±25.0% were observed for all levels greater than the DBS LOD. As both imprecision and bias results indicate an LOQ less than the observed LOD, the LOQ for DBS extracts is in practice equivalent to the LOD—0.180 U/mL (Clinical and Laboratory Standards Institute, EP17-A2, (2012)).

Investigation of the DBS assay's LOB, LOD, and LOQ indicates that a lower reporting limit can be achieved for DBS extracts. This may be attributable to the reduction of sample dependent matrix effects as a result of ˜tenfold extraction (dilution) with the approved assay diluent (Wood, W. G. ‘Matrix effects’ in immunoassays, Scand. J. Clin. Lab. Invest. Suppl., 205, 105-112 (1991)). As a result of the increased sensitivity, a clinical cutoff value below the EUA approved assay's LOQ for serum and plasma can be implemented for DBS extracts, enabling assessment of categorical agreement between venous and DBS samples near the serum cutoff (0.8 U/mL).

DBS Linearity

Linearity of the assay with DBS samples was assessed using 11 antibody concentrations made through serum sample admixtures in 10% increments followed by contrivance into DBS. Initial assessment of linearity indicated acceptable biases (±20.0%) from 0.0667 to 147 U/mL with 147 U/mL being the highest concentration tested. This study was repeated with samples of higher concentration to extend the measurement range to 250 U/mL in order to match the reporting limit of un-diluted samples for the EUA approved assay. For this second study, samples with extracted concentrations that initially measured >250 U/mL were manually diluted tenfold with Universal Diluent and re-measured as indicated in the assay's package insert (Roche Diagnostics, Elecsys Anti-SARS-CoV-2 S Elecsys Anti-SARS-CoV-2 S, (2020)). Antibody concentrations for this second study ranged from 0.0974 to 323 U/mL and targets were determined by using a linear fit of all data points.

DBS Clinical Cutoff

The clinical cutoff used to assign DBS results as negative or positive for antibodies for DBS samples was created using results from self-collected samples from donors confirmed to be seronegative (n=77). The standard deviation of these results (0.0405 U/mL) was multiplied by 3 and added to the mean (0.0625 U/mL) to give a value of 0.184 U/mL. The DBS clinical cutoff for positivity was then set to ≥0.185 U/mL, where all results less than 0.185 were reported as negative.

DBS Analytical Measurement Range

Evaluation of the detection capability of the assay with DBS extracts indicated that sample concentrations can be measured from 0.180 U/mL (LOD/LOQ) to 250 U/mL (upper reporting limit of assay) with a clinical negative/positive antibody cutoff concentration of 0.185 U/mL. This DBS range represents a calculated serum measurement range of about 2.6 to 3570 U/mL. Although dilution of samples is performed for venous serum and plasma samples for the EUA approved in order to extend the reporting limit, dilution of DBS samples is not currently utilized due to the relatively wider concentration range that can be reported for DBS samples (as a result of pre-dilution through extraction).

Imprecision of DBS Samples

Assay imprecision was assessed for DBS extracts using two reagent lots over a 4-day period with 6 contrived blood samples that covered a range of antibody concentrations (0.504-156 U/mL). A total of 16 replicates for each sample were measured. For both reagent lots, the CVs observed for repeatability and within-laboratory imprecision were less than 15.0/6 which meets FDA specifications for ligand binding assays (Table 1) (Food and Drug Administration. Bioanalytical method validation guidance for industry, Food Drug Adm., (2018)). All samples had a total categorical agreement of 100.0%.

TABLE 1

| | Mean | Repeatability | Within-Lab | Total |
|---|---|---|---|---|
| Sample | (U/mL) | (%) | (%) | Agreement |
| 1 | 0.530/0.504 | 7.6/6.1 | 10.9/9.4 | 100.0/100.0 |
| 2 | 0.682/0.661 | 8.5/7.6 | 11.3/10.1 | 100.0/100.0 |
| 3 | 5.97/5.93 | 7.7/7.6 | 14.7/12.8 | 100.0/100.0 |
| 4 | 15.3/15.3 | 6.9/6.1 | 8.6/8.0 | 100.0/100.0 |
| 5 | 61.8/61.9 | 12.5/13.2 | 12.5/13.2 | 100.0/100.0 |
| 6 | 156/156 | 7.7/7.9 | 8.7/7.9 | 100.0/100.0 |

B. Clinical Correlation Study

Following collection and measurement of samples, 6 of the 84 presumed negative donors had venous serum, self-collected DBS samples, and professionally collected DBS samples measure positive for SARS-CoV-2 antibodies despite having negative RT-PCR results at the time of serological specimen collection. The serum results for these donors ranged from 8.81 to 1170 U/mL where the clinical cutoff for serum samples was 0.800 U/mL. These results suggest previous (asymptomatic) infection or unreported vaccination. To confirm these results, the serum samples for these donors were measured using three additional EUA approved serology assays (Roche Elecsys Anti-SARS-CoV-2, DiaSorin Liaison SARS-CoV-2 S1/S2 IgG and DiaSorin Liaison SARS-CoV-2 IgM) (US Food & Drug Administration, EUA authorized serology test performance, FDA. 2, 1-14 (2020)). All six donors had serum results measure as positive on at least one additional assay and as such results were excluded from qualitative and quantitative analysis. All remaining results obtained were analyzed qualitatively using the established clinical cutoff for DBS extracts (0.185 U/mL) and quantitatively through correlative analysis.

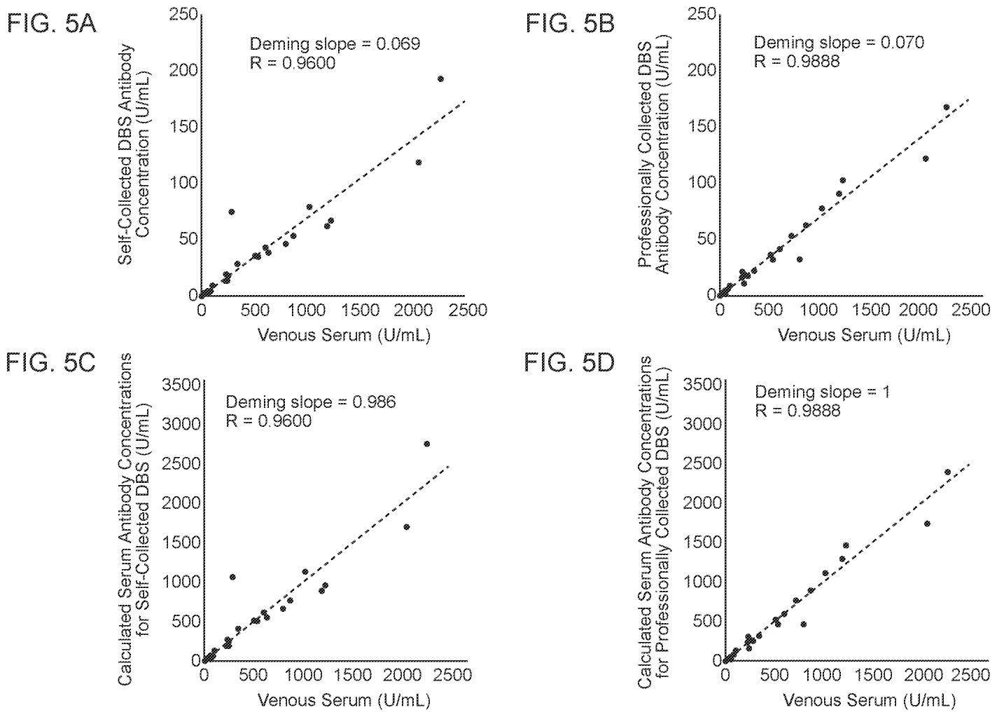

Comparison of serum samples with DBS obtained through self-collection demonstrated a high degree of agreement (R=0.9600) and Deming slope of 0.069 (n=108), which was attributed to dilution of the sample through the extraction process as well as precise but incomplete recovery of antibodies (FIGS. 5A and 5C). Two donors with negative venous serum results had self-collected DBS samples that measured positive for antibodies (results within 3.5× the DBS clinical cutoff). As a result of these two false positives as well as a false negative donor with unmeasurable antibody results for both serum and DBS, qualitative total categorical agreement of self-collected DBS samples compared to venous serum results was 98.1% (Table 2A). In addition, the negative percent agreement (NPA) and positive percent agreement PPA were found to be 97.4% and 100.0%, respectively. When compared to RT-PCR results, qualitative categorical agreement was 97.2% with an NPA and PPA of 97.3% and 97.1%, respectively (Table 2B). Results below and above the DBS measurement range were interpreted as negative and positive, respectively.

When quantitatively comparing professionally-collected DBS samples to serum results (n=106), results were similar to self-collected results with a correlation coefficient (R) of 0.9888 and a Deming slope of 0.070 (FIGS. 5B, and 5D). Total qualitative categorical agreement (n=111) to venous serum results was 100.0% (Table 2C). When compared to RT-PCR results, qualitative categorical agreement was 99.1% with an NPA and PPA of 100.0% and 97.0%, respectively (Table 2D). These results met FDA guidance at the time of these studies (NPA≥95.0%, PPA≥90.0%) (Food & Drug Administration website for Home Specimen Collection Serology Template for Fingerstick Dried Blood Spot, www.fda.gov/medical-devices/coronavirus-disease-2019-covid-19-emergency-use-authorizations-medical-devices/vitro-diagnostics-euas (2020)).

TABLE 2
Table 2A - Serum results

| Self-collected | | − | + | | |
|---|---|---|---|---|---|
| DBS | − | 74 | 0 | 74 | NPV − 100% |
| | + | 2 | 32 | 34 | PPV = 94.1% |
| | | 76 | 32 | 108 | Total |
| | | | | | Agreement |
| | | NPA = 97.4% | PPA = 100.0% | | 98.1% |

Table 2 B - RT-PCR results

| | | | | | |
|---|---|---|---|---|---|
| Self-collected | | − | + | | |
| DBS | − | 73 | 1 | 74 | NPV − 96.8% |
| | + | 2 | 33 | 35 | PPV = 94.3% |
| | | 75 | 34 | 109 | Total |
| | | | | | Agreement |
| | | NPA = 97.3% | PPA = 97.% | | 97.2% |

Table 2C- Serum results

| | | | | | |
|---|---|---|---|---|---|
| Professionally | | − | + | | |
| collected DBS | − | 79 | 0 | 79 | NPV − 100% |
| | + | 0 | 32 | 32 | PPV = 100,0% |
| | | 79 | 32 | 111 | Total |
| | | | | | Agreement |
| | | NPA = 100.0% | PPA = 100.0% | | 100.0% |

Table 2 D - RT-PCR results

| | | | | | |
|---|---|---|---|---|---|
| Professionally | | − | + | | |
| collected DBS | − | 78 | 1 | 79 | NPV − 98.7% |
| | + | 0 | 32 | 32 | PPV = 100.0% |
| | | 78 | 33 | 111 | Total |
| | | | | | Agreement |
| | | NPA = 100.0% | PPA = 97.0% | | 99.1% |

C. Robustness Studies

In order to evaluate sample stability during the shipping process, simulated shipping studies were performed in accordance with ISTA 7D guidance as recommended by the FDA (U. S. Food & Drug Administration. Home Specimen Collection Serology Template for Fingerstick Dried Blood Spot, (2020); International Safe Transit Association, Temperature Test for Transport Packaging; ISTA 7 Series Development Test Procedure, (2013)). Contrived DBS samples were prepared in triplicate and split between three storage conditions: room temperature (20-25° C.), a simulated winter and summer shipping excursions. Following completion of the excursions, samples were measured in a single measurement run where results from samples stored in parallel at room temperature were used as baseline results.

Additional robustness and analytical interference studies were performed to stress different aspects of the DBS sample collection process as well as the influence of several endogenous or exogenous interferences. For acceptance of results, a mean bias of ±20.0% was used as quantitative acceptance following the FDA guidance for ligand binding assays (Food and Drug Administration, Bioanalytical method validation guidance for industry. Food Drug Adm. (2018)). For qualitative assessment, a total categorical agreement of 95.0% was utilized based on guidance from the FDA's Home Specimen Collection Serology Template (U. S. Food & Drug Administration. Home Specimen Collection Serology Template for Fingerstick Dried Blood Spot, (2020)).

DBS Shipping Stability

For shipping excursion studies, contrived blood samples were created in triplicate and split into three shipping conditions: baseline ambient (20-25° C.), winter and summer excursions. Results less than 0.180 U/mL (DBS assay LOQ) were included in qualitative analysis but excluded from quantitative analysis. Overall, the sample results from winter and summer excursions both demonstrated total categorical agreement of 97.4% with mean biases of 6.0% and—0.5%, respectively (Table 3). These results indicate that samples are stable from the time of collection in an individual's home until received in the laboratory.

TABLE 3
Summary of DBS robustness study results.

| | | Mean | | Categorical | |
|---|---|---|---|---|---|
| Excursion | Hour(s) | Bias | n*: | Agreement | n: |
| Winter | 56 | 6.0% | 58 | 97.4% | 76 |
| Summer | 56 | −0.5% | 57 | 97.4% | 76 |
| Alternate Drying | 0 | −32.5% | 12 | 95.0% | 20 |
| Times | 1 | 1.3% | 12 | 95.0% | 20 |
| | 22 | 1.3% | 12 | 100.0% | 20 |
| Humid Drying | 1 | −13.3% | 12 | 95.0% | 20 |
| | 3 | −22.2% | 12 | 95.0% | 20 |
| | 22 | −44.6% | 12 | 95.0% | 20 |
| Contamination | | | | | |
| Alcohol Exposure | | −11.0% | 13 | 100.0% | 20 |
| Pressing Finger to Card | | 1.3% | 14 | 95.0% | 20 |

*Results below LOD were excluded from bias analysis

Stress Testing the Collection Process

For robustness studies, results that were found to be less than the assay's DBS LOQ were excluded from bias analysis but included in qualitative analysis. Investigation of drying times compared to the recommended drying time of 3 h (prior to sample storage) was performed. Samples that were immediately stored after contrived blood was added to the card had a categorical agreement of 95.0% and mean bias of—32.5%. Results from samples that were dried for 1 and 22 h demonstrated categorical agreements of 95.0 and 100.0%, respectively with mean bias of 1.3% for both (Table 3). Effects of drying samples in a humid environment (40° C., >95% relative humidity) were also investigated (U. S. Food & Drug Administration. Home Specimen Collection Serology Template for Fingerstick Dried Blood Spot, (2020)). All results observed had a categorical agreement of 95.0%, but the magnitude of the bias increased from—13.3% for 1 h to—44.6% for 22 h of humid drying.

Contamination as a result of potential errors in the collection process was investigated. Exposure of DBS spots to alcohol, which may occur following finger sterilization without allowing the fingertip to dry, demonstrated a categorical agreement of 100.0% and mean bias of—11.0%. Contamination of the DBS card by an unsterilized finger prior to collection demonstrated a categorical agreement of 95.0% and mean bias of 1.3%. Although all robustness studies had a total categorical agreement greater than or equal to 95.0%, biases less than ±20.0% were observed in some instances. As these studies are not exhaustive of all possible contaminants, proper instruction materials must be provided to the patient in order to insure the collection of a sample of sufficient quality for measurement.

Analytical Interference Studies

Several studies were performed to assess the effects of different endogenous and exogenous interferents on the measurement of SARS-CoV-2 antibodies from DBS samples. Results from all interference studies had a categorical agreement greater than or equal to 95.0% to baseline measurements (Table 4). The exogenous interferents tested had mean biases less than ±5.0% while most endogenous interferents tested had a mean bias result less than ±10.0% with the exception of excess protein (17.7%).

TABLE 4

| | | | | |
|---|---|---|---|---|
| Analytical | | | Categorical | |
| Interferents | Mean bias | N^{a} | agreement (%) | N |

Endogenous interferents

| | | | | |
|---|---|---|---|---|
| Hemolysis | 3.6 | 15 | 95.0 | 20 |
| (100%) | | | | |
| Triglycerides | 4.5 | 18 | 96.7 | 30 |
| (3000 mg/dL) | | | | |
| Total protein | 17.7 | 19 | 100.0 | 30 |
| (12 g/dL) | | | | |
| Conjugated | −2.8 | 20 | 96.7 | 30 |
| bilirubin | | | | |
| (20 mg/dL) | | | | |
| Unconjugated | −8.7 | 20 | 100.0 | 30 |
| bilirubin | | | | |
| (20 mg/dL) | | | | |

Exogenous interferents

| | | | | |
|---|---|---|---|---|
| Cerilliant mix 1 | −0.7 | 20 | 96.7 | 30 |
| Cerilliant mix 2 | 2.5 | 20 | 100.0 | 30 |
| Biotin | 4.5 | 21 | 96.7 | 29 |
| (3510 ng/ml) | | | | |

ªResults below LOD were excluded from bias analysis.

D. Immunization Study

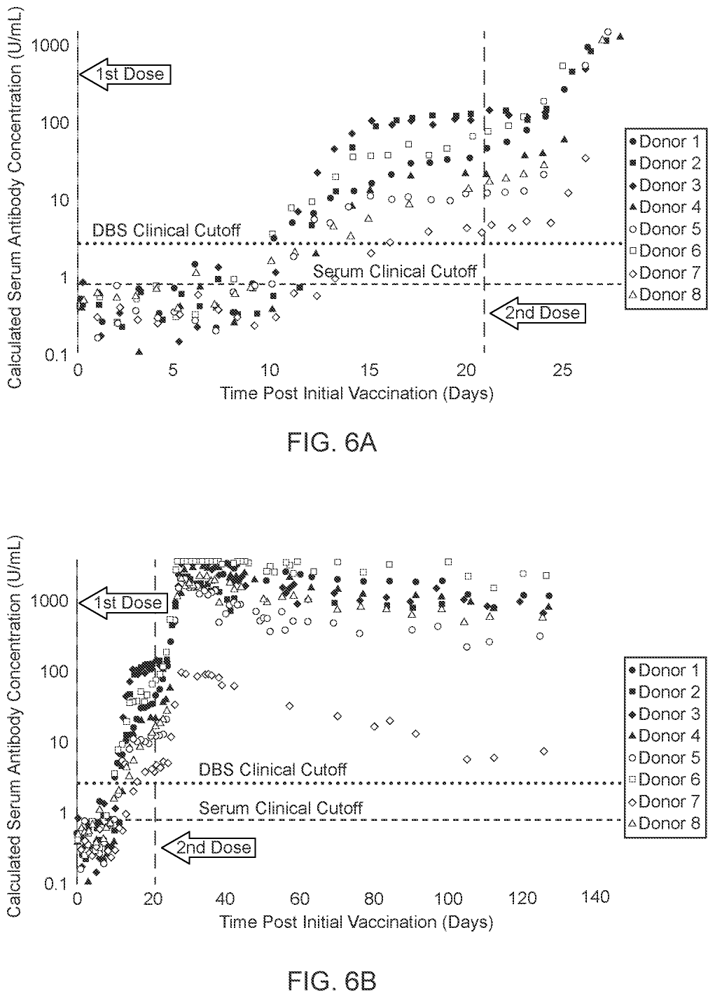

Application of self-collected DBS was performed with serial monitoring of SARS-CoV-2 antibody levels following immunization in 8 donors. Donors periodically performed self-collection of DBS samples pre-vaccination through 19 weeks following initial vaccination. All donors reported receiving the Pfizer-BioNTech COVID-19 vaccine with the second dose occurring exactly 3 weeks following the first dose. As can be seen in FIG. 6A all donors had negative DBS results for samples collected within the first 9 days following initial immunization. Antibody levels for each donor rose above the DBS cutoff of 0.185 U/mL between days 10 and 16. Antibody levels increased rapidly following the second vaccination dose (FIG. 6B), with several donors reaching levels greater than the DBS reporting limit (250 U/mL which is calculated to be 3570 U/mL in serum) (Livingston, E. H., Necessity of 2 doses of the Pfizer and Moderna COVID-19 vaccines, JAMA 325, 898 (2021); Dagan, N. et al., BNT162b2 mRNA COVID-19 vaccine in a nationwide mass vaccination setting, N. Engl. J. Med. 384, 1412-1423 (2021)). Dramatically lower antibody levels were observed for Donor 7, likely a result of the immunosuppressive medication the donor reported taking for a chronic condition (Negahdaripour, M. et al., Administration of COVID-19 vaccines in immunocompromised patients, Int. Immunopharmacol. 99, 108021 (2021)).

E. Conclusions

The results provided herein demonstrate the robustness of measuring SARS-CoV-2 antibodies using DBS samples. Although DBS samples are diluted through the extraction process (and as a result of incomplete antibody recovery), this approach also has advantages. Using Roche's Universal Diluent as the extraction buffer, sample-to-sample matrix effects were reduced and a lower (absolute) reporting limit was achieved (LOQ of 0.180 U/mL for DBS samples). In addition, the dilution of the sample allowed a higher relative measurement range to be demonstrated as DBS samples with a concentration of 250 U/mL DBS sample (upper limit of quantitation for undiluted venous samples) would be greater than 3500 U/mL in serum. These results, as well as the high correlation to venous serum results, allowed the assay to be used to demonstrate antibody monitoring over time through at-home DBS self-collections. Ultimately, DBS samples could serve as an important tool for regular antibody monitoring and scheduling of immunization boosters as antibody levels inferring protective immunity become more fully understood.

### Example 4—Embodiments

The disclosure may be better understood by referring to the following non-limiting embodiments. As used below, any reference to methods or systems is understood as a reference to each of those methods or systems disjunctively (e.g., “Illustrative embodiment 1-4 is understood as illustrative embodiment 1, 2, 3 or 4.”).

Illustrative embodiment 1 is a method for measuring an antibody in a dried sample comprising: (a) obtaining a sample from a subject; (b) extracting the antibody from the sample; and (c) detecting the antibody extracted from the sample.

Illustrative embodiment 2 is the method of any of the previous or subsequent method embodiments, wherein the antibody is an antibody to SARS-CoV-2.

Illustrative embodiment 3 is the method of any of the previous or subsequent method embodiments, wherein the sample is dried blood, or dried serum or dried plasma.

Illustrative embodiment 4 is the method of any of the previous or subsequent method embodiments, wherein the method is semi-quantitative or quantitative.

Illustrative embodiment 5 is the method of any of the previous or subsequent method embodiments, wherein the dried sample is a dried blood spot (DBS).

Illustrative embodiment 6 is the method of any of the previous or subsequent method embodiments, wherein the antibody is a SARS-COV-2 antibody and the step of detecting the SARS-COV-2 antibody is performed using a Roche Elecsys Anti-SARS-CoV-2 S electrochemiluminescence immunoassay.

Illustrative embodiment 7 is the method of any of the previous or subsequent method embodiments, wherein the LOD and/or LOQ is about 0.180 U/mL per DBS sample.

Illustrative embodiment 8 is the method of any of the previous or subsequent method embodiments, wherein the upper range of detecting or upper limit of quantitation (ULOQ) is about 250 U/mL DBS sample.

Illustrative embodiment 9 is the method of any of the previous or subsequent method embodiments, wherein the limit of blank (LOB) is about 0.111 U/mL per DBS sample.

Illustrative embodiment 10 is the method of any of the previous or subsequent method embodiments, wherein the DBS clinical cutoff for positivity is set to ≥0.185 U/mL, such that results less than 0.185 are reported as negative.

Illustrative embodiment 11 is the method of any of the previous or subsequent method embodiments, further comprising obtaining measurements from an individual over a period of time to follow the titer of SARS-CoV-2 antibody in the individual.

Illustrative embodiment 12 is the method of any of the previous or subsequent method embodiments, wherein the titer is followed in the individual for at least 19 weeks.

Illustrative embodiment 13 is the method of any of the previous or subsequent method embodiments, wherein the results are compared to RT-PCR detection of SARS-CoV-2 nucleic acid.

Illustrative embodiment 14 is the method of any of the previous or subsequent method embodiments, wherein compared to RT-PCR results, qualitative categorical agreement is about 99.1%, and/or a negative percent agreement (NPA) is 100.0%, and/or a positive percent agreement (PPA) is at least 97.0%.

Illustrative embodiment 15 is the method of any of the previous or subsequent method embodiments, wherein exogenous interferents have mean biases less than ±5.0%, and/or most endogenous interferents have a mean bias result less than ±10.0%.

Illustrative embodiment 16 is the method of any of the previous or subsequent method embodiments, wherein the antibody is measured using a sandwich assay employing a first antigen that recognizes the antibody and is labeled with a detectable moiety and a second antigen that recognizes the antibody and is labeled with a binding agent.

Illustrative embodiment 17 is the method of any of the previous or subsequent method embodiments, wherein the first and second antigens recognize antibodies to SARS-CoV-2 spike protein.

Illustrative embodiment 18 is the method of any of the previous or subsequent method embodiments, wherein the detectable moiety on the first antigen is an electrochemical moiety, and optionally, is ruthenium.

Illustrative embodiment 19 is the method of any of the previous or subsequent method embodiments, wherein the binding agent is streptavidin.

Illustrative embodiment 20 is the method of any of the previous or subsequent method embodiments, wherein the first antigen: SARS-COV-2 antibody: second antigen complex is bound to a biotin-labeled electrode such that application of a voltage results in a chemiluminescent emission.

Illustrative embodiment 21 is a system for performing any one of the previous method embodiments.

Illustrative embodiment 22 is a system for measuring an antibody in a dried sample comprising: (a) a component or station for obtaining a dried sample from a subject; (b) a component or station for extracting the antibody from the sample; and (c) a component or station detecting the antibody extracted from the sample.

Illustrative embodiment 23 is the system of any of the previous or subsequent system embodiments, wherein the antibody is an antibody to SARS-CoV-2.

Illustrative embodiment 24 is the system of any of the previous or subsequent system embodiments, wherein the sample is dried blood, or dried serum or dried plasma.

Illustrative embodiment 25 is the system of any of the previous or subsequent system embodiments, wherein detecting the antibody is semi-quantitative or quantitative.

Illustrative embodiment 26 is the system of any of the previous or subsequent system embodiments, wherein the dried sample is a dried blood spot (DBS).

Illustrative embodiment 27 is the system of any of the previous or subsequent system embodiments, wherein the antibody is a SARS-COV-2 antibody and detecting the SARS-COV-2 antibody is performed using a Roche Elecsys Anti-SARS-CoV-2 S electrochemiluminescence immunoassay.

Illustrative embodiment 28 is the system of any of the previous or subsequent system embodiments, wherein the LOD and/or LOQ is about 0.180 U/mL per DBS sample.

Illustrative embodiment 29 is the system of any of the previous or subsequent system embodiments, wherein the upper range of detecting or upper limit of quantitation (ULOQ) is about 250 U/mL DBS sample.

Illustrative embodiment 30 is the system of any of the previous or subsequent system embodiments, wherein the limit of blank (LOB) is about 0.111 U/mL per DBS sample.

Illustrative embodiment 31 is the system of any of the previous or subsequent system embodiments, wherein the DBS clinical cutoff for positivity is set to ≥0.185 U/mL, such that results less than 0.185 are reported as negative.

Illustrative embodiment 32 is the system of any of the previous or subsequent system embodiments, further comprising obtaining measurements from an individual over a period of time to follow the titer of SARS-CoV-2 antibody in the individual.

Illustrative embodiment 33 is the system of any of the previous or subsequent system embodiments, wherein the titer is followed in the individual for at least 19 weeks.

Illustrative embodiment 34 is the system of any of the previous or subsequent system embodiments, wherein the results are compared to RT-PCR detection of SARS-CoV-2 nucleic acid.

Illustrative embodiment 35 is the system of any of the previous or subsequent system embodiments, wherein compared to RT-PCR results, qualitative categorical agreement is about 99.1%, and/or the negative percent agreement (NPA) is 100.0%, and/or the positive percent agreement (PPA) is at least 97.0%.

Illustrative embodiment 36 is the system of any of the previous or subsequent system embodiments, wherein exogenous interferents have mean biases less than ±5.0%, and/or most endogenous interferents have a mean bias result less than ±10.0%.

Illustrative embodiment 37 is the system of any of the previous or subsequent system embodiments, wherein the antibody is measured using a sandwich assay employing a first antigen that recognizes the antibody and is labeled with a detectable moiety and a second antigen that recognizes the antibody and is labeled with a binding agent.

Illustrative embodiment 38 is the system of any of the previous or subsequent system embodiments, wherein the first and second antigens recognize antibodies to SARS-CoV-2 spike protein.

Illustrative embodiment 39 is the system of any of the previous or subsequent system embodiments, wherein the detectable moiety on the first antigen is an electrochemical moiety and optionally, is ruthenium.

Illustrative embodiment 40 is the system of any of the previous or subsequent system embodiments, wherein the binding agent is streptavidin.

Illustrative embodiment 41 is the system of any of the previous or subsequent system embodiments, wherein the first antigen: SARS-COV-2 antibody: second antigen complex is bound to a biotin-labeled electrode such that application of a voltage results in a chemiluminescent emission.

Illustrative embodiment 42 is a computer-program product tangibly embodied in a non-transitory machine-readable storage medium, including instructions configured to perform any of the steps of the previous method embodiments or run any part of the previous system embodiments.

Illustrative embodiment 43 is the computer-program product of any of the previous or subsequent computer-program product embodiments, including instructions configured to perform at least one of the steps of: (a) obtaining a sample from a subject; (b) extracting the antibody from the sample; and (c) detecting the antibody extracted from the sample.

Illustrative embodiment 44 is the computer-program product of any of the previous or subsequent computer-program product embodiments, including instructions configured to control or run at least in part at least one of: (a) a component or station for obtaining a dried sample from a subject; (b) a component or station for extracting the antibody from the sample; and (c) a component or station detecting the antibody extracted from the sample.

Illustrative embodiment 45 is the computer-program product of any of the previous or subsequent computer-program product embodiments, wherein the antibody is an antibody to SARS-CoV-2.

Illustrative embodiment 46 is the computer-program product of any of the previous or subsequent computer-program product embodiments, wherein the sample is dried blood, or dried serum or dried plasma.

That which is claimed is:

1. A method for measuring an antibody to in a dried sample from a subject comprising: (a) obtaining the dried sample from the subject; (b) extracting the antibody from the dried sample; and (c) detecting the antibody extracted from the dried sample.

2. The method of claim 1, wherein the antibody is a SARS-CoV-2 antibody.

3. The method of claim 1, wherein the step of detecting the antibody is semi-quantitative or quantitative.

4. The method of claim 1, wherein the dried sample is a dried blood spot (DBS).

5. The method of claim 1, wherein the dried sample is dried plasma or dried serum.

6. The method of claim 1, wherein the assay is an electrochemiluminescence immunoassay.

7. The method of claim 2, wherein the antibody is measured using a sandwich assay employing a first antigen that recognizes the SARS-CoV-2 antibody and is labeled with a detectable moiety and a second antigen that recognizes the SARS-CoV-2 antibody and is labeled with a binding agent.

8. The method of claim 7, wherein the first and second antigens recognize antibodies to SARS-CoV-2 spike protein.

9. The method of claim 7, wherein the detectable moiety on the first antigen is an electrochemical moiety and optionally, is ruthenium.

10. The method of claim 7, wherein the binding agent is streptavidin.

11. The method of claim 7, wherein the first antigen: SARS-CoV-2 antibody: second antigen complex is bound to a biotin-labeled electrode such that application of a voltage results in a chemiluminescent emission.

12. The method of claim 11, wherein the sample is a dried blood spot, and the limit of detection (LOD) and/or lower limit of quantitation (LLOQ) for the SARS-CoV-2 antibody is 0.180 U/mL per dried blood spot sample.

13. The method of claim 11, wherein the sample is a dried blood spot, and the upper limit of quantitation (ULOQ) for the SARS-CoV-2 antibody is 250 U/mL per dried blood spot sample.

14. The method of claim 11, wherein the sample is a dried blood spot, and the dried blood spot clinical cutoff for positivity for the SARS-CoV-2 antibody is set to ≥0.185 U/mL.

15. The method of claim 2, further comprising obtaining measurements from an individual over a period of time to follow the titer of SARS-CoV-2 antibody in the individual.

16. A system for measuring an antibody in a dried sample comprising: (a) a component or station for obtaining a dried sample from a subject; (b) a component or station for extracting the antibody from the sample; and (c) a component or station for detecting the antibody extracted from the sample.

17. The system of claim 16, wherein the antibody is a SARS-CoV-2 antibody.

18. The system of claim 16, wherein detecting the antibody extracted from the sample step is semi-quantitative or quantitative.

19. The system of claim 16, wherein the dried sample is a dried blood spot (DBS), dried plasma or dried serum.

20. A computer-program product tangibly embodied in a non-transitory machine-readable storage medium, including instructions configured to run at least in part at least one of: (a) a component or station for obtaining a dried sample from a subject; (b) a component or station for extracting the antibody from the sample; and (c) a component or station detecting the antibody extracted from the sample.
